SEQUENCE LISTING (10 sequences)

SEQ ID NO: 1 - protein, 327 aa, Homo sapiens
GCCCTGTGGATGCGCCTCCTGCCCCTGCTGGCGCTGCTGGCCCTCTGGGGACCTGACCCAGCCGCAGCCUUUGUGAACCAACACCUGUGCGGCUCACACCUGGUGGAAGCUCUCUACCUAGUGUGCGGGGAACGAGGCUUCUUCUACACACCCAAGACCCGCCGGGAGGCAGAGGACCUGCAGGUGGGGCAGGUGGAGCUGGGCGGGGGCCCUGGUGCAGGCAGCCUGCAGCCCUUGGCCCUGGAGGGGUCCCUGCAGAAGCGUGGCAUUGUGGAACAAUGCUGUACCAGCAUCUGCUCCCUCUACCAGCUGGAGAACUACUGCAAC

SEQ ID NO: 2 - protein, 16 aa, Homo sapiens
GGGGSGGGGSGGGGGS

SEQ ID NO: 3 - protein, 11 aa, Homo sapiens
FYVEQSTFSRH

SEQ ID NO: 4 - protein, 30 aa, Homo sapiens
EAAAKEAAAKEAAAKEAAAKEAAAKEAAAK

SEQ ID NO: 5 - protein, 13 aa, Homo sapiens
QLSPFPFDLNLGK

SEQ ID NO: 6 - protein, 11 aa, Homo sapiens
DICLPRWGCLW

SEQ ID NO: 7 - protein, 394 aa, Homo sapiens
EDPQGDAAQKTDTSHHDQDHPTFNKITPNLAEFAFSLYRQLAHQSNSTNIFFSPVSIAAAFAMLSLGAKGDTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 8 - protein, 12 aa, Homo sapiens
THRPPMWSPVWP

SEQ ID NO: 9 - protein, 254 aa, Homo sapiens
ALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNGGGGSGGGGSGGGGSFYVEQSTFSRHEAAAKEAAAKEAAAKEAAAKEAAAKEAAAKQLSPFPFDLNLGKGGGGSGGGGSGGGGSDICLPRWGCLWEAAAKEAAAKEAAAKEAAAKEAAAKEAAAKMDPVPVPPVPVPMPSTNGGA

SEQ ID NO: 10 - RNA, 761 nt, Synthetic construct
  source 1..761: mol_type = unassigned RNA
gccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaacggcggcggcggctctggcggcggcggctctggcggcggcggctctttttatgtggaacagtctaccttttctcgcacgaagcggcggcgaaagaagcggcggcgaaagaagcggcggcgaaagaagcggcggcgaaagaagcggcggcgaaagaagcggcggcgaaacagctgagcccgtttccgtttgatctgaacctgggcaaaggcggcggcggctctggcggcggcggctctggcggcggcggctctgatatttgcctgccgcgctggggctgcctgtgggaagcggcggcgaaagaagcggcggcgaaagaagcggcggcgaaagaagcggcggcgaaagaagcggcggcgaaagaagcggcggcgaaaatggatccggtgccggtgccgccggtgccggtgccgatgccgagcaccaacggcggcgcg

(19) United States
(12) Patent Application Publication
(10) Pub. No.: US 2026/0265802 A1
(43) Pub. Date: Sep. 10, 2026

(54) LOW-COST CELL-FREE INSULIN BASED ON SELF AMPLIFYING RNA

(71) Applicant: DEI Biopharma, Kampala (UG)

(72) Inventor: Matthias Magoola, Kampala (UG)

(21) Appl. No.: 19/072,942

(22) Filed: Mar. 6, 2025

(51) Int. Cl. C12P 21/02; A61K 38/00; C07K 14/62

(52) U.S. Cl. CPC C12P 21/02; C07K 14/62; A61K 38/00; C07K 2319/30; C07K 2319/31

(57) ABSTRACT

The claimed technology relates to a method of manufacturing long-acting insulin in a cell-free system using self-amplifying linear or circular RNA, yielding the said insulin fused with multiple short peptides known to bind with long half-life endogenous proteins to enhance the half-life of insulin resulting in reduced frequency of administration of insulin; the cell-free RNA expression yields 1,000 to 1,000,000 molecules of insulin for each RNA molecule, reducing the manufacturing size and reduces the cost of goods (COGs) to less than the cost of the packaging of insulin.

### SEQUENCE LISTING

The instant application contains a Sequence Listing presented per 37 C.F.R. 1.822, which has been submitted electronically in ASCII format and is hereby incorporated by reference in its entirety. Said ASCII text file, created on Feb. 28, 2025, is named LOW-COST CELL-FREE INSULIN.xml, and is 11 kb in size.

### TECHNICAL FIELD

The claimed technology relates to a method of manufacturing long-acting insulin in a cell-free system using self-amplifying linear or circular RNA, yielding the said insulin fused with multiple short peptides known to bind with long half-life endogenous proteins to enhance the half-life of insulin resulting in reduced frequency of administration of insulin; the cell-free RNA expression yields 1,000 to 1,000,000 molecules of insulin for each RNA molecule, reducing the manufacturing size and reduces the cost of goods (COGs) to less than the cost of the packaging of insulin.

### BACKGROUND

The following description of the background of the present technology is provided simply as an aid in understanding the present technology. It is not admitted that it describes or constitutes prior art to the present technology.

Fusion proteins combining therapeutic molecules with Fc domains, albumin, or transferrin have emerged as a strategy to extend plasma half-life and improve pharmacokinetic properties; in the case of fusion with Fc domains, it is a result of interaction with the neonatal Fc receptor (FcRn), facilitating recycling and prolonging circulation time; in other instances, it is the half-life of the conjugated protein. Additional factors include receptor binding affinity, intracellular processing, and linker design (Table 1) in extending the half-life of the fusion protein (Table 1).

TABLE 1
Endogenous proteins and their half-lives

| | Nominal | | The ratio of | | UniProt |
|---|---|---|---|---|---|
| | Half-Life | Molecular | Half-Life to | Half-Life | Accession |
| Protein | (hours) | Mass (kDa) | Molecular Mass | Determinant | Number |
| Elastin | 682,560 | 70 | 9,750 | Extremely | P15502 |
| | | | | long-lived | |
| | | | | structural | |
| | | | | protein | |
| Collagen | 21,024 | 300 | 70 | Long-lived | P02452 |
| | | | | structural | |
| | | | | protein | |
| IgG1 | 480 | 146 | 3.3 | FcRn recycling | P01857 |
| IgG2 | 480 | 146 | 3.3 | FcRn recycling | P01859 |
| IgG4 | 480 | 146 | 3.3 | FcRn recycling | P01861 |
| Albumin | 456 | 67 | 6.8 | Size-dependent | P02768 |
| Transferrin | 288 | 80 | 3.6 | Receptor- | P02787 |
| | | | | mediated | |
| | | | | recycling | |
| Factor XIII | 168 | 320 | 0.5 | Size-dependent | P00488 |
| IgG3 | 144 | 165 | 0.87 | FcRn recycling | P01860 |
| Pentameric | 144 | 970 | 0.15 | Receptor | P01871 |
| IgM | | | | interactions | |
| | | | | and size | |

### DETAILS OF THE INVENTION

To enhance the half-life of insulin, it can be fused with the listed proteins in Table 1. However, an alternate solution is to fuse insulin with short peptides known to bind with these endogenous proteins (Table 1) to secure the same impact while allowing multiple bindings with different endogenous proteins in a much smaller size of the target drug with half-life extended. Table 2 lists the short peptides that can bind to endogenous long half-life proteins.

TABLE 2
List of Short peptide sequences for fusion protein structuring

Component: SEQUENCE
Proinsulin Sequence (UniProt: P01308-1) (SEQUENCE NO. 1):
GCCCTGTGGATGCGCCTCCTGCCCCTGCTGGCGCTGCTGGCCCTCTGGGGACCTGACCCA
GCCGCAGCCUUUGUGAACCAACACCUGUGCGGCUCACACCUGGUGGAAGCUCUCUACC
UAGUGUGCGGGGAACGAGGCUUCUUCUACACACCCAAGACCCGCCGGGAGGCAGAGGA
CCUGCAGGUGGGGCAGGUGGAGCUGGGCGGGGGCCCUGGUGCAGGCAGCCUGCAGCCC
UUGGCCCUGGAGGGGUCCCUGCAGAAGCGUGGCAUUGUGGAACAAUGCUGUACCAGC
AUCUGCUCCCUCUACCAGCUGGAGAACUACUGCAAC
(Gly₄Ser)₃ Flexible Linker (SEQUENCE NO. 2):
GGGGSGGGGSGGGGGS
Fc-Binding Peptide 1 (Synthetic Peptide) (SEQUENCE NO. 3):
FYVEQSTFSRH
(EAAAK)₅ Rigid Linker (SEQUENCE NO. 4)
EAAAKEAAAKEAAAKEAAAKEAAAKEAAAK
Fc-Binding Peptide 2 (FcRn Mimic) (SEQUENCE NO. 5):
QLSPFPFDLNLGK
Albumin-Binding Peptide (SEQUENCE NO. 6)
DICLPRWGCLW
α1-Antitrypsin (AAT) Domain (PDB: 1KCT) (SEQUENCE NO. 7):
EDPQGDAAQKTDTSHHDQDHPTFNKITPNLAEFAFSLYRQLAHQSNSTNIFFSPVSIAAAFAM
LSLGAKGDTHDEILEGLNFNLTEIPEAQIHEGFQELLRTLNQPDSQLQLTTGNGLFLSEGLKLV
DKFLEDVKKLYHSEAFTVNFGDTEEAKKQINDYVEKGTQGKIVDLVKELDRDTVFALVNYIF
FKGKWERPFEVKDTEEEDFHVDQVTTVKVPMMKRLGMFNIQHCKKLSSWVLLMKYLGNAT
AIFFLPDEGKLQHLENELTHDIITKFLENEDRRSASLHLPKLSITGTYDLKSVLGQLGITKVFSN
GADLSGVTEEAPLKLSKAVHKAVLTIDEKGTEAAGAMFLEAIPMSIPPEVKFNKPFVFLMIEQ
NTKSPLFMGKVVNPTQK
Transferrin Synthetic Peptide (SEQUENCE NO. 8):
THRPPMWSPVWP
Expressed Protein (SEQUENCE NO. 9):
ALWMRLLPLLALLALWGPDPAAAFVNQHLCGSHLVEALYLVCGERGFFYTPKTRREAEDLQ
VGQVELGGGPGAGSLQPLALEGSLQKRGIVEQCCTSICSLYQLENYCNGGGGSGGGGSGGGG
SFYVEQSTFSRHEAAAKEAAAKEAAAKEAAAKEAAAKEAAAKQLSPFPFDLNLGKGGGGSG
GGGSGGGGSDICLPRWGCLWEAAAKEAAAKEAAAKEAAAKEAAAKEAAAKMDPVPVPPV
PVPMPSTNGGA
Open Reading Frame RNA Sequence: (SEQUENCE NO. 10):
GCCCTGTGGATGCGCCTCCTGCCCCTGCTGGCGCTGCTGGCCCTCTGGGGACCTGACCCA
GCCGCAGCCTTTGTGAACCAACACCTGTGCGGCTCACACCTGGTGGAAGCTCTCTACCTA
GTGTGCGGGGAACGAGGCTTCTTCTACACACCCAAGACCCGCCGGGAGGCAGAGGACCT
GCAGGTGGGGCAGGTGGAGCTGGGCGGGGGCCCTGGTGCAGGCAGCCTGCAGCCCTTGG
CCCTGGAGGGGTCCCTGCAGAAGCGTGGCATTGTGGAACAATGCTGTACCAGCATCTGCT
CCCTCTACCAGCTGGAGAACTACTGCAACGGCGGCGGCGGCTCTGGCGGCGGCGGCTCT
GGCGGCGGCGGCTCTTTTTATGTGGAACAGTCTACCTTTTCTCGCACGAAGCGGCGGCGA
AAGAAGCGGCGGCGAAAGAAGCGGCGGCGAAAGAAGCGGCGGCGAAAGAAGCGGCGG
CGAAAGAAGCGGCGGCGAAACAGCTGAGCCCGTTTCCGTTTGATCTGAACCTGGGCAAA
GGCGGCGGCGGCTCTGGCGGCGGCGGCTCTGGCGGCGGCGGCTCTGATATTTGCCTGCCG
CGCTGGGGCTGCCTGTGGGAAGCGGCGGCGAAAGAAGCGGCGGCGAAAGAAGCGGCGG
CGAAAGAAGCGGCGGCGAAAGAAGCGGCGGCGAAAGAAGCGGCGGCGAAAATGGATCC
GGTGCCGGTGCCGCCGGTGCCGGTGCCGATGCCGAGCACCAACGGCGGCGCG

To ensure the protein-binding peptide remains attached after proinsulin processing, it must be placed outside the C-peptide region, which is enzymatically removed during insulin maturation. The two main locations for conjugation are at the N-terminal of the B-chain or the C-terminal of the A-chain.

By adding the protein-binding peptide at the start of the B-chain, insulin's overall function is minimally affected, and it remains albumin-bound post-cleavage. This placement allows for extended circulation of insulin without interfering with insulin receptor binding.

Alternatively, the protein-binding peptide can be placed at the end of the A-chain, ensuring it remains part of the mature insulin molecule. This placement preserves normal insulin folding and maintains full biological activity while allowing albumin binding.

In some examples, the C-terminus of the insulin polypeptide is connected directly to the N-terminus of the long half-life endogenous proteins (e.g., no linker or linker absent). In other examples, successfully constructing a recombinantly made instant invention insulin requires a linker connecting the insulin polypeptide to the long half-life endogenous proteins. In embodiments, the instant invention insulin configurations described herein comprise a peptide linker between the insulin polypeptide and the long half-life endogenous proteins composed of amino acids (e.g., natural or unnatural amino acids).

In other embodiments, the peptide linker can be encoded by a nucleic acid molecule, for example, such that a single nucleic acid molecule can encode the various peptides within an insulin polypeptide as well as the peptide linker and the long half-life endogenous proteins. The choice of peptide linker (for example, the length, composition, hydrophobicity, and secondary structure) could impact the manufacturability. The linker selection is based on their extensive structural and functional requirements:
GGPPS linkers: Incorporate proline residues for proteolytic stability while maintaining flexibility, showing a 3-fold reduced degradation compared to GGGGS linkers.
EAAAK helical spacers: Form rigid α-helical structures that provide defined spatial separation between domains, crucial for independent function.
GGGGS flexible linkers: Allow proper domain movement where required, particularly around the insulin molecule, to maintain receptor binding.

A modeling exercise is required to ensure the validity of insulin bound with endogenous proteins, such as transferrin, and is not hindered in binding to insulin receptors. The structure of the insulin-insulin receptor complex has been documented in the Protein Data Bank (PDB ID: 6PXV). The methodology involved pre-processing and standardizing the available insulin-receptor structure using UCSF Chimera and predicting the structure of human serum transferrin attached to insulin via a (G4S)1 linker using AlphaFold3. Subsequently, insulin was docked onto the receptor's binding site using HADDOCK in its native form and after linkage with transferrin through the (G4S)1 linker. Binding affinity and interaction patterns were evaluated using the PRODIGY server, focusing on the effect of the transferrin attached to insulin via a linker on interaction characteristics and steric hindrance.

The results of protein-protein complex analysis revealed no significant differences in binding affinity and interaction characteristics (Table 3). For the native insulin-insulin receptor complex, the ΔG value was calculated at −16.7 kcal mol⁻¹, with a corresponding kD of 5.3 E⁻¹³ (Table 1). Interface analysis demonstrated a balanced contribution of polar-polar and apolar-apolar interactions within the binding pocket. Upon conjugation with transferrin via the (G4S)1 linker, we observed slightly enhanced binding affinity and similar interaction profiles. The Transferrin-(G4S)1-Insulin-Receptor complex exhibited a relatively higher ΔG value (−16.9 kcal mol⁻¹) and the lowest kD (4e−^{13 M}), indicating stronger binding and enhanced stability. Interface analysis further revealed significant increases in polar-polar and apolar-apolar interactions for the Transferrin-(G4S)1 conjugate, while charged-charged interactions remained consistent across all models. Additionally, the conjugation of transferrin to insulin with the (G4S)1 linker would result in slower dissociation in the body, improving the pharmacokinetic profile and making it a better therapeutic product (Table 3).

TABLE 3
Binding properties of insulin with receptor when conjugated with transferrin.

| Protein- | ΔG | | ICs | ICs | ICs | ICs | ICs | ICs | | |
|---|---|---|---|---|---|---|---|---|---|---|
| protein | (kcal | | charged- | charged- | charged- | polar- | polar- | apolar- | NIS | NIS |
| complex | mol⁻¹) | Kd (M) | charged | polar | apolar | polar | apolar | apolar | charged | apolar |
| Insulin | −16.7 | 5.30e−13 | 19 | 10 | 61 | 2 | 16 | 31 | 28.54 | 34.3 |
| Receptor | | | | | | | | | | |
| Transferrin- | −16.9 | 4.00e−13 | 23 | 10 | 61 | 2 | 16 | 31 | 29.87 | 34.4 |
| G4S- | | | | | | | | | | |
| Insulin | | | | | | | | | | |
| receptor | | | | | | | | | | |

Insulin treatment requiring less frequent dosing (e.g., once-weekly or once-monthly, once bi-monthly, once every six months, or even yearly injections) would be less burdensome, leading to better compliance, glucose control, and ultimately better long-term health outcomes.

The conjugation or fusion of insulin with Fc, albumin, or transferrin significantly influences its half-life and dosing requirements. Unconjugated insulin has a rapid clearance with a half-life of only 5-10 minutes, necessitating frequent administration to maintain therapeutic levels. In contrast, albumin-insulin fusion can extend the half-life to ~21 days in humans, allowing for much less frequent dosing, sometimes as little as once daily. Similarly, transferrin-insulin fusion can provide target-specific action, such as brain delivery, with an extended half-life determined by tissue recycling dynamics (Table 4).

TABLE 4
Comparison of Insulin Half-Life Extension
by Albumin, Fc of IgG, and Transferrin

| Conjugation or | | Expected | | |
|---|---|---|---|---|
| fusion Strategy | Half-Life Mechanism | Half-Life | | |
| Regular Insulin | Rapid clearance | ~4-6 | min | |
| Insulin-Albumin | FcRn recycling | ~5-7 days | | 21 days |
| | reduced renal | (avg 6) | | |
| | clearance | | | |
| Insulin-Fc of IgG | FcRn recycling, | ~10-21 days | | 50 days |
| | delayed clearance | (avg 14) | | |
| Insulin-Fc1 and | Maximum recycling | 30+ | days | 110 days |
| Fc2 + albumin + | and degradation | | | |
| transferase | protection | | | |

| | | | |
|---|---|---|---|
| Insulin- | TfR-mediated | ~7-14 days | 36 days |
| Transferrin | transport & recycling, | (avg 10) | |
| | brain penetration | | |

The dosing frequency will depend on the nature of the protein conjugated and the type of RNA expression system used. To achieve a target of 60 units of insulin in the body, insulin conjugated with a protein that yields a half-life of 14 days for the conjugate and administered as an amplifying RNA that completes the amplifying cycle in 7 days that will be achieved if a single 60 units of RNA are administered yielding 7.87 units per day. The RNA is administered every 50 days; a repeat dose earlier than 50 days can be established based on the insulin concentration in any given patient. The administration can be arranged through an insulin pump or an inserted device that will yield a specified quantity based on the patient's needs. Using this ratio, Table 5 makes projections of the frequency of insulin dosing.

Table 5. Dosing of RNA (Proinsulin-Fc) with Regular Insulin.

| | | | | Adjusted |
|---|---|---|---|---|
| | | | | Ratio |
| | Approximate | Ratio to | Molecules/ | (Expression- |
| RNA Type | MW (Da) | Insulin | RNA molecule | Weighted) |
| Insulin | 5800 | 1 | n/a | 1 |
| Linear mRNA | 405000 | 69.7 | 200-800 | 0.14 |
| Self-amplifying | 3350000 | 576.8 | 10,000-50,000 | 0.023 |
| mRNA | | | | |
| circRNA | 460000 | 79.2 | 1,000-6000 | 0.02 |
| Self-amplifying | 3400000 | 585.4 | 75,000-700,000 | 0.0017 |
| circRNA | | | | |

Substantially smaller doses of RNA product will be needed compared to the quantity of insulin injected. Broad calculations show that the cost of an RNA product is likely to be less than the cost of the product's packaging, such as dispensing it in a syringe dosage form.

### Cell-Free Expression

While the long-acting insulin will reduce the cost of treatment substantially, it will remain unaffordable to billions of patients across the globe. To reduce this burden, the instant invention produces its long-acting insulin using a self-amplifying RNA in a cell-free system that can reduce the cost of goods to less than the cost of its packaging, making insulin affordable to all patients across the globe.

The instant invention insulin suitable for an ultra-long-acting therapy for diabetes can be manufactured by any recombinant technology, chemically conjugating insulin with protein-binding peptides or RNA technology, preferably using a self-amplifying RNA in a cell-free system. Table 4 lists the selected short peptides to fuse with proinsulin.

The development of long-acting insulin analogs represents a significant advancement in diabetes treatment. Here, we present a novel multi-component design that combines circulatory protein binding, matrix interaction, and stability enhancement strategies to achieve unprecedented half-life extension.

5′ Cap→5′ UTR→Replicase (nsP1-nsP4)→Subgenomic Promoter→Target ORF (Proinsulin fused with short peptides)→3′ UTR→Poly(A) Tail

nsP1 (Nonstructural Protein 1): UniProt Entry: P36327 (involved in capping the viral RNA, crucial for RNA stability and recognition by the host's translational machinery); nsP2 (Nonstructural Protein 2): UniProt Entry: P36328 (possesses helicase and protease activities; involved in processing the nonstructural polyprotein and mode; nsP3 (Nonstructural Protein 3): UniProt Entry: P36329 (plays a role in viral RNA replication and modulation of host cell processes); nsP4 (Nonstructural Protein 4): UniProt Entry: P36330 (serves as the RNA-dependent RNA polymerase, essential for replicating the viral RNA genome).

The subgenomic promoter from VEEV has a sequence of UAGUACUUUAACAAUU. The full design of the linear saRNA structure is shown in Table 6

TABLE 6
Linear saRNA structure

| Component | Optimized Sequence |
|---|---|
| 5′ Cap | m7GpppG |
| Optimized 5′ UTR | GGGAGCCACCAGGAAUAA |
| (SEQUENCE NO. 11) | ACUAGUAUUCUUCUGGUC |
| | CCCACAGACUCAGAGA |
| Replicase (nsP1-nsP4) | UniProt Entry: P36327 |
| VEEV, UniProt | UniProt Entry: P36328; |
| | UniProt Entry P36329; |
| | UniProt Entry: P36330 |
| Subgenomic Promoter | UAGUACUUUAACAAUU |
| (SGP) from VEEV | |
| (SEQUENCE NO. 12) | |
| Start Codon | AUG |
| Target ORF | Proinsulin Sequence |
| | (UniProt: P01308-1) |
| | plus short-peptide |
| | fused with linkers |
| Stop Codon | UAA |
| Optimized 3′ UTR | AAGGCGGCGCCUAUGUUA |
| (SEQUENCE NO. 13) | CGUGCAAAGGUGAUUGUC |
| | ACCCCCCAGUGCGGUAUG |
| | GAUACGAAUCCUUUCUUG |
| | CCGUUAAUUGGAUUGGAA |
| | CUCAUUUGACGAUUCGAA |
| | GAAAGAGAGGAGAAAUCA |
| | UUUGUUUUU |
| Polyadenylation Signal | GAAUCGGAUUUUGUUUUU |
| (SEQUENCE NO. 14) | AAUAUUUC |
| Poly (A) Tail | AAAAAAAAAAAAAAAAAA |
| (SEQUENCE NO. 15) | AAAAAAAAAAAAAAAAAA |
| | AAAAAAAAAAAAAA |

To express long-acting insulin, an optimal selection involves two Fc peptides, one albumin peptide, and one α1-Antitrypsin domain attached to the proinsulin sequence.

The protein sequence is determined by the following embodiment as an example: Proinsulin Sequence (UniProt: P01308-1)→(Gly₄Ser)3 Flexible Linker→Fc-Binding Peptide 1 (Synthetic Peptide)→(EAAAK)₅→Fc-Binding Peptide 2 (FcRn Mimic)→(Gly₄Ser)₃ Flexible Linker→Albumin-Binding Peptide→(EAAAK)₅→α1-Antitrypsin (AAT) Domain (PDB: 1KCT). The expression ratios are presented in Table 7.

TABLE 7
Expression ratios of proinsulin molecules

| | Linear mRNA, | Circular RNA, |
|---|---|---|
| Property | self-amplifying | self-amplifying |
| Expression | 10,000x-1,000,000x | 100,000x-10,000,000x |
| Ratio | (high amplification | (high stability + |
| | due to replication) | self-replication) |
| Duration of | 48-72 hours | 7-10 days |
| expression | | |

While traditional cell-free protein synthesis (CFPS) systems are widely available, commercially available lysates specifically optimized for saRNA expression are limited. However, several CFPS platforms may be adapted for saRNA applications, such as E. coli-Based Systems PURExpress® provided by New England Biolabs. Thermo Fisher Scientific supplies Expressway; wheat germ extracts are suitable for eukaryotic protein expression; and Promega is used; Rabbit Reticulocyte Lysates. Thermo Fisher Scientific: Human Cell Lysates are derived from human cells and offer human-like post-translational modifications. 1-Step Human Coupled IVT Kit: Thermo Fisher Scientific; Insect Cell Lysates: Thermo Fisher Scientific; CHO Cell Lysates: Creative Biolabs.

Adapting these systems for saRNA expression may require optimization, as saRNA replication necessitates specific factors not inherently present in standard lysates. Supplemented with additional components or engineer lysates to support RNA replication mechanisms.

The production of insulin using self-amplifying RNA (saRNA) in a CHO lysate-based cell-free system involves a multi-step process, beginning with the design and synthesis of linear saRNA encoding proinsulin.

The saRNA construct includes a T7 promoter, 5′ UTR, replicase (nsPs for amplification), proinsulin ORF, 3′ UTR, and a poly(A) tail to enhance stability and translation efficiency. The plasmid DNA is first amplified in E. coli, purified, and used as a template for in vitro transcription (IVT) using T7 RNA polymerase, rNTPs, and a cap analog such as CleanCap. The transcribed RNA is then purified using LiCl precipitation and silica column methods, followed by quality control checks through agarose gel electrophoresis, Nanodrop/Qubit quantification, and Bioanalyzer analysis to confirm integrity.

CHO lysate is prepared for cell-free protein synthesis by culturing CHO-K1 or CHO-S cells, lysing them via freeze-thaw cycles or homogenization, and centrifuging to remove debris. The lysate is then combined with a translation buffer containing HEPES (pH 7.4), potassium acetate, magnesium acetate, ATP, GTP, creatine phosphate, amino acids, and a tRNA mix, followed by the addition of saRNA at one μg per 50 μL reaction volume. The reaction is incubated at 30-37° C. for 1-6 hours, optimizing conditions to maximize protein synthesis. Protein expression is monitored using Western blot (anti-insulin antibodies) and HPLC-MS to confirm the presence of proinsulin.

To convert proinsulin into active insulin, prohormone convertases (PC1/3 and PC2) and carboxypeptidase E (CPE) are added to the CHO lysate reaction, followed by incubation at 37° C. for 2-4 hours. Proper disulfide bond formation is ensured using protein disulfide isomerase (PDI) and a redox buffer containing GSH: GSSG (1:5 ratio, 1-5 mM total). The insulin is then purified through ion-exchange chromatography (IEC) using a DEAE-Sepharose column, followed by size-exclusion chromatography (SEC) using a Superdex 75 column to remove aggregates and impurities. Quality control testing includes HPLC analysis to assess purity, mass spectrometry to confirm molecular weight and cleavage efficiency, and glucose uptake assays in adipocytes or muscle cells to verify bioactivity.

The final formulation involves adjusting the pH to 7.2-7.4, sterile filtration (0.2 μm), and lyophilization for storage, with the purified insulin stored at −20° C. to −80° C. For scalability, IVT can be expanded using bioreactors and automated continuous chromatography systems for large-scale purification. GMP compliance requires stringent batch documentation, sterility validation, and endotoxin testing. This CHO lysate-based insulin production method eliminates the need for bacterial refolding steps and allows direct post-translational processing, making it an efficient, scalable, and high-yield alternative to traditional recombinant insulin manufacturing.

1. A method of producing long-acting, low-cost insulin comprising fusing proinsulin with a plurality of short peptides capable of binding with long half-life endogenous proteins with a flexible linker and expressing by cell-free self-amplifying RNA (saRNA).

2. The method of producing long-acting, low-cost insulin of claim 1, wherein short-peptides to bind proinsulin comprise Fc-Binding Peptide 1 (Synthetic Peptide) (SEQUENCE NO. 3): FYVEQSTFSRH, Fc-Binding Peptide 2 (FcRn Mimic) (SEQUENCE NO. 5): QLSPFPFDLNLGK, Albumin-Binding Peptide (SEQUENCE NO. 6) DICLPRWGCLW, and Transferrin Synthetic Peptide (SEQUENCE NO. 8): THRPPMWSPVWP, or a combination thereof.

3. The method of producing long-acting, low-cost insulin of claim 1, wherein long half-life endogenous proteins comprise albumin, Fc Region (IgG), and transferrin or a combination thereof.

4. The method of producing long-acting, low-cost insulin of claim 1, wherein the structure of expression protein comprises proinsulin Sequence (UniProt: P01308-1)→(Gly₄Ser)3 Flexible Linker→Fc-Binding Peptide 1 (Synthetic Peptide)→(EAAAK)₅→Fc-Binding Peptide 2 (FcRn Mimic)→(Gly₄Ser)₃ Flexible Linker→Albumin-Binding Peptide→(EAAAK)₅→α1-Antitrypsin (AAT) Domain (PDB: 1KCT).

5. The method of producing long-acting, low-cost insulin of claim 1, wherein the expressed protein is
(SEQUENCE NO. 9)
ALWMRLLPLLALLALWGPDPAAAFVNQHLCGSHLVEALYLVCGE
RGFFYTPKTRREAEDLQVGQVELGGGPGAGSLQPLALEGSLQKR
GIVEQCCTSICSLYQLENYCNGGGGSGGGGSGGGGSFYVEQSTF
SRHEAAAKEAAAKEAAAKEAAAKEAAAKEAAAKQLSPFPFDLNL
GKGGGGSGGGGSGGGGSDICLPRWGCLWEAAAKEAAAKEAAAKE
AAAKEAAAKEAAAKMDPVPVPPVPVPMPSTNGGA.

6. The method of producing long-acting, low-cost insulin of claim 1, wherein the RNA Open Reading Frame is:
(SEQUENCE NO. 10)
CCAGCCGCAGCCUUUGUGAACCAACACCUGUGCGGCUCACACCU
GGUGGAAGCUCUCUACCUAGUGUGCGGGGAACGAGGCUUCUUCU
ACACACCCAAGACCCGCCGGGAGGCAGAGGACCUGCAGGUGGGG
CAGGUGGAGCUGGGCGGGGGCCCUGGUGCAGGCAGCCUGCAGCC
CUUGGCCCUGGAGGGGUCCCUGCAGAAGCGUGGCAUUGUGGAAC
AAUGCUGUACCAGCAUCUGCUCCCUCUACCAGCUGGAGAACUAC
UGCAACGGCGGCGGCGGCUCUGGCGGCGGCGGCUCUGGCGGCGG
CGGCUCUUUUUAUGUGGAACAGUCUACCUUUUCUCGCACGAAGC
GGCGGCGAAAGAAGCGGCGGCGAAAGAAGCGGCGGCGAAAGAAG
CGGCGGCGAAAGAAGCGGCGGCGAAAGAAGCGGCGGCGAAACAG
CUGAGCCCGUUUCCGUUUGAUCUGAACCUGGGCAAAGGCGGCGG
CGGCUCUGGCGGCGGCGGCUCUGGCGGCGGCGGCUCUGAUAUUU
GCCUGCCGCGCUGGGGCUGCCUGUGGGAAGCGGCGGCGAAAGAA
GCGGCGGCGAAAGAAGCGGCGGCGAAAGAAGCGGCGGCGAAAGA
AGCGGCGGCGAAAGAAGCGGCGGCGAAAAUGGAUCCGGUGCCGG
UGCCGCCGGUGCCGGUGCCGAUGCCGAGCACCAACGGCGGCGCG.

7. The method of producing long-acting, low-cost insulin of claim 1, wherein the flexible linkers comprise GGPPS, GGGGS and EAAAK, single or multiples of sequence or a combination thereof.

8. The method of producing long-acting, low-cost insulin of claim 1, wherein the saRNA is linear or circular.

9. The method of producing long-acting, low-cost insulin of claim 5, wherein saRNA expression is made in fortified extracts or lysates from E. coli, Wheat Germ, Rabbit Reticulocyte, Human Cells, Insect Cells, and CHO Cell, or a combination thereof.

10. The method of producing long-acting, low-cost insulin of claim 5, wherein the open reading frame includes an α-trypsin SEQUENCE NO. 7.

11. The method of claim producing long-acting, low-cost insulin of claim 5, wherein each molecule of RNA expresses 100 to 1,000,000 molecules of proinsulin.

12. The method of claim producing long-acting, low-cost insulin of claim 1, wherein the long-acting proinsulin is injected weekly, monthly, or yearly.

13. The method of claim producing long-acting, low-cost insulin of claim 1, wherein the long-acting proinsulin is injected through an insulin pump.

14. The method of claim producing long-acting, low-cost insulin of claim 1, wherein the fused proinsulin is administered daily, weekly, monthly, bimonthly, every six months, or yearly, and a combination thereof.

15. The method of claim producing long-acting, low-cost insulin of claim 1, wherein the cost of goods (COGs) of fused proinsulin 1000 units is less than the cost of its packaging.
